(19) United States
(12) Patent Application Publication
(10) Pub. No.: US 2021/0290563 A1
(43) Pub. Date: Sep. 23, 2021

(54) COMPOSITIONS AND METHODS FOR TREATING NEUROPSYCHIATRIC DISEASES

(71) Applicant: BIONICHE GLOBAL DEVELOPMENT, LLC, McLean, VA (US)

(72) Inventors: DAVID GORDON DANIEL, MCLEAN, VA (US); NOAH JACKSON GORDON DANIEL, MCLEAN, VA (US); DONALD THEODORE GORDON DANIEL, MCLEAN, VA (US)

(73) Assignee: BIONICHE GLOBAL DEVELOPMENT, LLC, MCLEAN, VA (US)

(21) Appl. No.: 16/678,966

(22) PCT Filed: Nov. 8, 2019
(86) PCT No.: PCT/US2018/031562

§ 371 Date: Nov. 8, 2019

Related U.S. Application Data
(60) Provisional application No. 62/504,170, filed on May 10, 2017.

(51) Int. Cl. A61K 31/05; A61K 45/06; A61K 9/08; A61K 31/661; A61P 25/24; A61P 25/18; A61P 25/22; A61K 9/00

(52) U.S. Cl. CPC A61K 31/05; A61K 45/06; A61K 9/08; A61K 31/661; A61P 25/24; A61K 9/0014; A61P 25/22; A61K 9/006; A61K 9/0019; A61K 9/0043; A61K 9/0053; A61P 25/18

(57) ABSTRACT

This invention relates to compositions and methods for treating or ameliorating symptoms of a neuropsychiatric disease in a subject in need thereof comprising: (a) a therapeutically effective amount of propofol; and (b) a therapeutically effective amount of one or more psychotropic drug and a pharmaceutically acceptable carrier or diluent.

### CROSS REFERENCE TO RELATED APPLICATIONS

This application is the National Stage of International Application No. PCT/US2018/031562, filed May 8, 2018, which claims priority to U.S. Provisional Application No. 62/504,170, filed May 10, 2017, the entire content of which is incorporated herein by reference.

### FIELD OF THE INVENTION

Described herein is the use of propofol in the treatment of neuropsychiatric diseases.

### I. BACKGROUND OF THE INVENTION

Neuropsychiatric diseases are a general class of diseases that focus on brain disorders with cognitive and behavioral abnormalities presented with somatic signs as well as those disorders of mood and thought associated with no, or minor physical signs of neurological abnormalities and the motor and sensory systems. Schizophrenia, depression, anxiety disorders, and autism are examples of neuropsychiatric diseases.

According to the World Health Organization (WHO), by 2030, the amount of disability and life lost as a result of depression will be greater than that resulting from any other condition, including accidents, war, suicide, cancer, and stroke. Suicide is the third most common cause of death in adolescents, behind traffic accidents and deaths from HIV/AIDS. Close to 800,000 die due to suicide every year and there are many more who attempt suicide. Hence, many millions of people are affected or experience suicide bereavement every year. Suicide occurs throughout the lifespan and is the second leading cause of death globally.

Depression is one of the main drivers of suicide, which takes the lives of 30,000 Americans yearly. Among those hospitalized for depression, perhaps up to 15 percent will go on to die of suicide. Major depressive disorder (MDD) has become psychiatry's signature diagnosis. Depression is diagnosed in about 40% of patients who see a psychiatrist. This percentage is double that of just 20 years ago, and it is far higher than that of any other diagnosis.

Consumption of antidepressants has soared since 1990. Roughly 10% of women and 4% of men in the United States take antidepressant medication at any time. In 21^{st} century, antidepressants were the best-selling prescription drugs of any type. Yet epidemiological studies suggest that there are still vast numbers of untreated depressed individuals. Antidepressants have been around since the 1950s. The number of compounds has multiplied, but there has been no corresponding increase in their efficacy, and there has been an increase in the number of treatment-resistant depression. Accordingly, pharmaceutical companies have pulled back on drug development in this field in recent years.

Treatment-resistant depression (TRD) or treatment-refractory depression is a term used in clinical psychiatry to describe cases of major depressive disorder (MDD) that do not respond adequately to appropriate courses of at least two antidepressants. Inadequate response has traditionally been defined as no response whatsoever. However, many clinicians consider a response inadequate if the patient does not achieve full remission of symptoms.

There are a vast number of other psychiatric disorders for which there are no approved FDA medications. There is no report in the literature for an effective therapeutic regimen addressing underlying diseases or symptoms of diseases related to autism, including Asperger's Syndrome, and other related disorders including diminished comfort in social interactions and diminished social initiative, Pervasive Developmental Disorder, Social Phobia and Negative Symptoms of Schizophrenia, Post-Traumatic Stress Disorder (PTSD), and catatonic disorders including but not limited to catatonia associated with another mental disorder or medical condition or unspecified Catatonia among others.

The majority of Neuropsychiatric drugs currently marketed create dependence on subject patients which in turn has caused serious and deleterious effects on the social, economic and medical condition of the patient. The problem with drug addiction is specifically compounded by the ease of access to several addictive drugs that are readily available and routinely used by large segments of population. Medical and non-medical use of prescription pain relievers, sedatives, and stimulants are also on the increase. The addictive ability of drugs has been linked to their pharmacological actions on mesotelencephalic dopamine (DA) reinforcement/reward pathways in the central nervous system (CNS). Dopaminergic transmission within these pathways is modulated by gamma-amino butyric acid (GABA).

Accordingly, there has been much interest in the scientific community in attempting to find substances that could be employed to treat or ameliorate symptoms of neuropsychiatric diseases. It is therefore clear that there has been and remains today a long-standing need for compositions and methods for early treatment of neuropsychiatric diseases and related disorders before the disease manifests far enough to produce permanent psychological changes, and thereby allowing earlier and more effective therapeutic intervention. Accordingly, there is a long-felt need for discovering new compositions and methods that can achieve such therapeutic effects in patients with neuropsychiatric diseases or related disorders with a short or a long-time treatment schedules without causing concomitant neuropsychiatric drug dependency. The present invention as disclosed and described herein addresses this need and solves the problem.

### II. SUMMARY OF THE INVENTION

The present invention is directed to compositions and methods for treating neuropsychiatric diseases and related disorders in a subject in need thereof.

In its broadest aspect, methods for treating or ameliorating symptoms of a neuropsychiatric disease are provided that requires administering to a subject in need thereof a therapeutically effective amount of propofol.

In another aspect, a pharmaceutical composition for treating neuropsychiatric diseases are provided comprising: (a) a therapeutically effective amount of a first composition comprising a propofol composition; and (b) a therapeutically effective amount of a second composition comprising one or more psychotropic drugs. The first composition and the second composition are each administered together or separately with a pharmaceutically acceptable carrier or diluent.

In one embodiment of the present invention, the propofol composition comprises, propofol analogues, derivatives, salts and racemic mixtures, and or prodrugs and metabolites of propofol or fospropofol disodium or any combination thereof.

Neuropsychiatric diseases within the scope of the invention include depression, suicidality, Post-Traumatic Stress Disorder (PTSD), Asperger's syndrome, Pervasive Developmental Disorder, Social Phobia, anhedonia and Negative Symptoms of Schizophrenia, anorexia, and catatonic disorders including but not limited to catatonia associated with another mental disorder or medical condition or unspecified Catatonia.

According to one embodiment of the invention, the one or more psychotropic drugs comprise Selective Serotonin Reuptake Inhibitors (SSRIs), Serotonin and Norepinephrine Reuptake Inhibitors (SNRIs), Tricyclic Antidepressants, Monoamine Oxidase Inhibitors, antipsychotic medication, anticonvulsants, mood stabilizing medication, stimulants, hypnotic agent, benzodiazepines, and ketamine, or any combination thereof.

In another embodiment, propofol and/or propofol in combination with one or more psychotropic drug is administered for the treatment of an acute neuropsychiatric disease, maintenance of the patient suffering from such diseases and prevention of the replace of such neuropsychiatric diseases.

According to another embodiment of the invention, propofol and one or more psychotropic drugs are formulated in one or different solutions and they can also be formulated in a dry or liquid form.

According to yet another embodiment of the invention, propofol and one or more psychotropic drugs are administered contemporaneously or at different time intervals.

According to one aspect of the invention, a method for treating or ameliorating symptoms of a neuropsychiatric disease is provided that requires administrating to a subject in need thereof: (a) a therapeutically effective amount of propofol; and (b) a therapeutically effective amount of one or more psychotropic drug and a pharmaceutically acceptable carrier or diluent.

According to one embodiment of the invention, the therapeutic method treats depression instantaneously and/or rapidly.

The mode of administration and the dosage of propofol and the one or more psychotropic drugs are determined on the basis of the clinical situation at hand and are tailored on the basis of the individual patient's needs. In general, propofol and the one or more psychotropic drugs can be administered orally, parenterally, transdermally, intranasally, or intravenously. Propofol and the one or more psychotropic drugs or both can also be administered in a time-released manner.

In yet another aspect of the invention, there is provided a kit for treating or ameliorating one or more symptoms of a neuropsychiatric disease. The kit includes, a) a first composition comprising a propofol composition; b) a second composition comprising one or more psychotropic drugs; and c) instructions for the use of the first and second compositions.

In a further aspect of the invention, there is provided a method of achieving an enhanced therapeutic effectiveness of a psychotropic drug in a subject in need of treatment thereof comprising administrating one or more psychotropic drug in combination with a propofol composition, wherein the effective therapeutic concentration of said one or more psychotropic drug is reduced by about 10-100 fold when administered with the propofol composition as compared to a control group that did not receive the propofol composition.

The reduced dosing of the psychotropic drug results in prevention or amelioration of one or more side effects resulted from the prolonged use of the psychotropic drugs. The side effect of prolonged usage of these drugs include addiction, dysphoria, anxiety, panic, impairment of memory, reductions in psychomotor and cognitive performance, disordered perception of the passage of time, euphoria, schizophrenic psychosis, tiredness, dizziness, tachycardia, orthostatic hypotension, dry mouth, reduced lacrimation, muscle relaxation, increased appetite, potential irreversible cognitive impairments, or any combination thereof.

### III. DETAILED DESCRIPTION OF THE INVENTION

Reference now will be made in detail to embodiments of the invention. It will be apparent to those skilled in the art that various modifications and variations can be made in the present invention without departing from the scope or spirit of the invention. For instance, features illustrated or described as part of one embodiment, can be used on another embodiment to yield a still further embodiment.

In general, the present invention is directed to compositions and methods related to the use of propofol or fospropofol for the treatment of depression including, treatment resistant depression, the rapid treatment of depression, and the treatment of acute and chronic suicidality, as well as the treatment of Post-Traumatic Stress Disorder (PTSD), Asperger's Syndrome, pervasive developmental disorder, social phobia, anxiety, anorexia, anhedonia, schizophrenia, and catatonic disorders including but not limited to catatonia associated with another mental disorder or medical condition or unspecified Catatonia or any combination thereof.

The therapy of the present invention has unexpectedly completely circumvented the known deleterious side effects associated with the use of current antidepressants. Current treatments for depression fail to provide substantive relief in many patients and when effective typically take 2 to 4 weeks for onset of significant antidepressant effects. In addition, patients who fail their initial treatment often do not respond to subsequent trials, and frequently experience a course of illness marked by chronic depression, impaired psychosocial functioning, and poor overall general health. For example, in a well-known study of 3671 outpatients known as STAR-D, remission rates were 36.8%, 30.6%, 13.7%, and 13.0% for the first, second, third, and fourth sequential attempts at treatment.

The administration of propofol with one or more psychotropics according to this invention not only would be effective in treating the underlying disorder but would also be supportive in the control of the usage and dosage of the psychotropics, as well as other accompanied drugs, such as opioids, narcotics, cannabinoids, and general class of pain medications, that are addictive by nature. This combination would result in reducing the dosage and period of administration of the psychotropics as well other accompanied drugs by reducing their side effects in the patients under therapy and thereby reducing the risk of addiction and other undesirable side effects.

Propofol, as used herein, refers to fospropofol disodium, analogues, derivatives, salts and racemic mixtures, prodrugs and/or metabolites of propofol or fospropofol disodium.

Psychotropic drug, as used herein, refers to a wide range of drugs that affect neurological or phycological symptoms in patients. Psychotropic drugs within the scope of this invention include, by way of example and not limitation, antidepressant medications, antipsychotic medications, mood stabilizing medications, stimulants, hypnotic agent, benzodiazepines, ketamine, among others.

Propofol (2,6-diisopropylphenol) is an intravenous sedative/hypnotic agent used extensively for induction and maintenance of general anesthesia, sedation of critically ill patients and procedural sedation. Propofol is only sparingly soluble in water. Propofol is a GABAA agonist that activates multiple GABAA receptor subtypes, which are ion channels that transport chlorine anions across cell membranes, in the central nervous system, Although propofol is achiral, racemic mixtures of a number of dialkyl phenols are known agonists of the GABAA receptor and it has been found that propofol to be superior in its overall profile to other analogues evaluated.

Propofol is commonly administered in ambulatory settings because of its rapid onset, dose-related hypnotic effect, rapid recovery and favorable safety profile. It is typically formulated in an oil-in-water emulsion and is highly lipophilic, with a rapid onset and short duration of action. The pharmacokinetics of propofol are described by a three-compartment linear model with compartments representing the plasma, rapidly equilibrating tissues such as the brain, and slowly equilibrating tissues such as adipose.

Following an IV bolus dose, there is rapid equilibration between the plasma and highly perfused brain tissue, which accounts for the rapid onset of anesthesia. Rapid redistribution accounts for its short duration of action.

Propofol's mode of action is considered distinctive from other anesthetic agents. It has no direct affinity for benzodiazepine or N-Methyl D Aspartate (NMDA) receptors. Propofol's anesthetic effects are felt to derive principally from modulating the inhibitory function of the neurotransmitter gamma-aminobutyric acid (GABA) through GABA-A receptors. However, propofol also inhibits NMDA receptors, reduces calcium influx through slow calcium channels, and possesses antioxidant, immunomodulatory and anticonvulsant activity. In addition, propofol inhibits Ach release from the frontal cortex and hippocampus.

Propofol is preferred by many clinicians due to its excellent pharmacokinetic, pharmacodynamic, emergence and recovery profiles. However, undesired side-effects (e.g., respiratory depression, airway collapse, ICU syndrome, injection pain and hemodynamic effects) produced at or near the therapeutic dose greatly limit its utility in multiple clinical settings.

According to one embodiment of the present invention, Propofol is used in combination with electroconvulsive therapy (ECT) in the treatment of major depressive disorder (MDD).

Higher dose administration of psychotropics can be avoided by co-administration of a propofol composition and one or more psychotropics, whereby increasing the bioavailability and improving the adsorption rate of the psychotropics and their passage through cell membranes and the blood brain barrier. An unlimiting example of psychotropic drugs, by way of example but not limitation, include antidepressants, antipsychotic, mood stabilizing medications, stimulants, hypnotic agents, antiepileptic, anti-anxiety, anti-inflammatory, sedatives, benzodiazepines, ketamine, among other psychotropics.

In one embodiment, the therapeutic method of the invention is a combination therapy that includes propofol as well as a second composition of one or more psychotropic drugs. psychotropic drugs are a heteromorphic group of chemicals that act on different receptors on cells that regulate press neurotransmitter release in the brain. These receptor proteins include the endopsychotropics (produced naturally in the body by humans and animals), the phytopsychotropics (found in cannabis and some other plants), and synthetic psychotropics (manufactured chemically).

There are many side effects associated with psychotropic drugs and especially to the higher dosages of these drugs and with their long treatment administration. The present invention provides a combination therapy of one or more psychotropic drug and propofol, wherein the overall therapeutic benefits of both the psychotropic drug and propofol are increased considerably. Furthermore, the reduction or prevention of the side effects of psychotropic drug when administered in combination with a propofol composition provides an unexpected synergistic benefit.

Psychotropics according to this invention can either be exogenic or endogenic in origin. Exogenic psychotropics can be both natural, and synthetic. These exogenic psychotropics can be both binding, aka agonists and non-binding, aka antagonists to neuronal cells and their receptors. For example, endogenic psychotropics (i.e. Anandamide, 2-AG, etc.) and endogenous receptors sites (i.e. CB1 and CB2) are found throughout the body and regulate homeostasis in a wide variety of physiological and neurological functions from birth till death. Endopsychotropics are found in the human placenta and even breast milk and are essential to all the body's regulatory functions.

What follows is a non-limiting listing of the psychotropics that may be used in the combination therapy compositions and methods of the present invention. Sertraline (Zoloft), fluoxetine (Prozac), citalopram (Celexa), escitalopram (Lexapro), paroxetine (Paxil, Pexeva), fluvoxamine (Luvox), trazodone (Oleptro), Desvenlafaxine (Pristiq), Duloxetine (Cymbalta), Venlafaxine (Effexor XR), Atomoxetine, Mirtazapine, Amitriptyline, Amoxapine, Clomipramine (Anafranil), Desipramine (Norpramin), Doxepin, Imipramine (Tofranil), Nortriptyline (Pamelor), Trimipramine (Surmontil), Isocarboxazid (Marplan), Phenelzine (Nardil), Selegiline (Emsam), Tranylcypromine (Parnate), Lurasidone, Quetiapine, Brexpiprazole, Lamotrigine, Ketamine, GLYX13 Rapastinel, 2. NRX 1074, Memantine, CERC 301, Traxoprodil, and Cycloserine, among others.

The novel combination of a propofol composition with one or more psychotropics, which are synergistic when used in combination, results in easy delivery of and passage of a propofol composition and psychotropics through the cell membrane and the blood brain barrier. The effect of the inventive therapy is manifested by a slowing of the progression of one or more of the symptoms of a neuropsychiatric diseases including, for example, and not by way of limitation, disturbed sleep, nausea, dizziness, difficulty walking, blurred vision, impaired memory, cognitive impairment, inhibition of neurogenesis, brain damage, brain shrinkage, blackouts, severe anxiety, tremors, major depression, dysthymia, mania, hypomania, panic disorder, suicidality, Post-traumatic Stress Disorder, Asperger's Syndrome, pervasive developmental disorder, social phobia, anorexia, anhedonia, anxiety and schizophrenia.

In one embodiment, this invention is directed to a first composition for use with a second composition for ameliorating symptoms of a neuropsychiatric disease in a subject suffering from such disease, wherein said first composition comprises (a) a therapeutically effective amount of a propofol composition; and (b) a therapeutically effective amount of a second composition comprising one or more psychotropics, a natural or synthetic derivative thereof, or a salt thereof, wherein said first composition and said second composition are each optionally and independently administered together with a pharmaceutically acceptable carrier or diluent.

As a result of the administration of a propofol composition according to the present invention, the transport of one or more psychotropics through cell membranes and the blood brain barrier is further facilitated. Because of the ease and efficiency of transport of psychotropics that is caused by use of a propofol composition, the effective concentration of psychotropics can be reduced by as much as about 10-fold to about 100-fold or more without reducing the therapeutic effectiveness of this drug.

The optimization of dosing of psychotropics achieved with the compositions and methods of the present invention has tremendous clinical advantages in preventing the one or more side effects of use of psychotropics including, for example, and not by away of limitation those effects such as dysphoria, and anxiety or panic, impairment of memory, reductions in psychomotor and cognitive performance, disordered perception of the passage of time, and euphoria, schizophrenic psychosis, tiredness, dizziness, tachycardia, orthostatic hypotension, dry mouth, muscle relaxation, potential irreversible cognitive impairments or any combination thereof that are known side effects of any psychotropic-based therapy.

The psychotropics for use in the compositions and methods of the present invention are isolated endogenous psychotropics, Phyto psychotropics, recombinant psychotropics, or a combination thereof, or can be administered exogenously, or may be administered through a combination of both endogenous and exogenous sources of psychotropics.

The terms “treat”, “treated”, “treating” and “treatment” are to be broadly understood as referring to any response to, or anticipation of, a medical condition in a mammal, particularly a human, and includes but is not limited to: (i) preventing the medical condition from occurring in a subject, which may or may not be predisposed to the condition, but has not yet been diagnosed with the condition and, accordingly, the treatment constitutes prophylactic treatment for the medical condition; (ii) inhibiting the medical condition, i.e., arresting, slowing or delaying the onset, development or progression of the medical condition; or (iii) relieving the medical condition, i.e., causing regression of the medical condition.

The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

It will be understood that a therapeutically and/or prophylactically effective amount of a drug for a subject is dependent inter alia on the body weight of the subject as well as other factors known to a person of ordinary skill in the art. A “subject” herein to which a therapeutic agent or composition thereof can be administered includes mammals such as a human subject of either sex and of any age.

As used herein, an “effective amount” of a composition is an amount sufficient to achieve a desired biological effect, in this case at least one of amelioration or treatment of symptoms of a neuropsychiatric disease that are targeted by the combination therapy of the invention. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. In one embodiment, the therapeutically effective amount of the composition of the present invention is used via a pharmaceutical carrier, a liposome, a micelle, or small unilamellar vesicle (SUV) for the entrapment of the therapeutically effective amount of one or more psychotropics. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

The active compositions of the invention having activities as described herein are provided as isolated and substantially purified compounds in pharmaceutically acceptable formulations using formulation methods known to those of ordinary skill in the art. These formulations can be administered by standard routes.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In general, the combinations may be administered by the transdermal, intraperitoneal, intracranial, transcranial delivery, intracerebroventricular, intracerebral, intravaginal, intrauterine, oral, rectal, ophthalmic (including intravitreal or intracameral), nasal, olfactory endothelium, topical (including buccal and sublingual), parenteral (including subcutaneous, intraperitoneal, intramuscular, intravenous, intradermal, intracranial, intratracheal, and epidural and nasal) administration. Parenteral administration includes direct or indirect injection into cells, tissues or organs in vivo, ex vivo or in vitro.

In one embodiment, the combination therapy comprising use of a first composition comprising a propofol composition and the second composition comprising one or more psychotropics is administered through one or more different or the same routes of administration in a single or multiple regimen. In one embodiment, first composition comprising a propofol composition and the second composition comprising one or more psychotropics can be administered by a variety of routes and modes of administration, including for example, and not by way of limitation, intravenous routes, transdermal routes, intranasal routes, parenteral routes, oral routes or a combination thereof. In one embodiment, the first composition comprising a propofol composition and the second composition comprising one or more psychotropics is administered once, twice, three, four or more times daily through, IV routes, oral routes, or a combination of both.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided. The compositions are administered separately or are mixed together prior to administration.

In one embodiment, the first composition, the second composition or both may be incorporated into biodegradable polymers allowing for sustained release of the compound, the polymers being implanted in the vicinity of where drug delivery is desired or implanted so that the composition is slowly released systemically. Osmotic minipumps may also be used to provide controlled delivery of the first composition, the second composition or both through cannula to the site of interest, such as directly into the site of injury. The biodegradable polymers and their use are described, for example, in detail in Brem et al., J. Neurosurg. 74: 441-446 (1991), which is hereby incorporated by reference in its entirety.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The composition formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

A typical regimen for treatment of symptoms of diseases and disorders related to neuropsychiatric diseases comprises administration of an effective amount of the composition as described above, administered as a single treatment, or repeated as enhancing or booster dosages, over a period up to and including one week to about 48 months or more.

Within other embodiments, the compositions may also be placed in any location such that the compounds or constituents are continuously released. The amount of the composition of the invention which will be effective in the treatment of symptoms of diseases and disorders related to neuropsychiatric diseases can be determined by standard clinical techniques. In addition, in vitro assays may optionally be employed to help identify optimal dosage ranges. In particular, the dosage of the compositions of the present invention will depend on the disease state of subject under treatment and other clinical factors such as weight and condition of the human or animal and the route of administration of the compounds or compositions. The precise dose to be employed in the formulation, therefore, should be decided according to the judgment of the health care practitioner and each patient's circumstances. Effective doses may be extrapolated from dose-response curves derived from in vitro or animal model test systems.

Various delivery systems are available and can be used to administer the compositions of the invention, i.e., encapsulation in liposomes, microparticles, microcapsules, recombinant cells capable of expressing the compound, receptor-mediated endocytosis. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (i.e., oral mucosa, rectal and intestinal mucosa, etc.) and may be administered together with other biologically active agents. Administration can be systemic or local.

In addition, it may be desirable to introduce the compounds or compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the compositions of the invention locally to the area in need of treatment; this may be achieved by, for example, and not by way of limitation, local infusion during surgery, or topical application.

In one embodiment, the compound or composition can be delivered in a controlled release system. In one embodiment, a pump may be used. In another embodiment, polymeric materials can be used. In yet another embodiment, a controlled release system can be placed in proximity of the target, i.e., the brain, thus requiring only a fraction of the systemic dose. Other controlled release systems are discussed in the review by Langer, Science 249:1527-1533 (1990).

In yet another embodiment, the compositions of the invention are administered via aerosolization including but not limited to thermal aerosolization.

Preferred unit dosage formulations are those containing a daily dose or unit, daily sub-dose, or an appropriate fraction thereof, of the administered ingredient. It should be understood that in addition to the ingredients, particularly mentioned herein, the formulations of the present invention may include other agents conventional in the art having regard to the type of formulation in question.

Non-limiting representative examples of various dosage ranges for the first composition comprising a propofol composition and the second composition comprising one or more psychotropics are as follows. In one embodiment, propofol is administered at a dosage of about 0.1 mg/kg, 0.3 mg/kg, 1 mg/kg, 2 mg/kg, 5 mg/kg, 10 mg/kg, 15 mg/kg, or 20 mg/kg, or any numerical integer values there between, at a weekly or biweekly interval. In another embodiment, psychotropics disclosed herein are administered at a dosage of about 1 mg/kg to about 400 mg/kg. For example, about 5 mg/kg, 10 mg/kg, 15 mg/kg, 20 mg/kg, 50 mg/kg, 60 mg/kg, 100 mg/kg, 150 mg/kg, 200 mg/kg, 250 mg/kg, or 400 mg/kg, or any numerical integer values there between, on a daily, weekly or a biweekly interval.

As would be understood by one of ordinary skill in the art, when a composition of the present invention is provided to an individual, it can further comprise at least one of salts, buffers, adjuvants, or other substances which are desirable for improving the efficacy of the composition. Adjuvants are substances that can be used to specifically augment at least one immune response. Normally, the adjuvant and the composition are mixed prior to presentation to the immune system or presented separately.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Adjuvants can be generally divided into several groups based upon their composition. These groups include lipid micelles, oil adjuvants, mineral salts (for example, AlK(SO₄)₂, AlNa (SO₄)₂, AlNH₄ (SO₄)), silica, kaolin, and certain natural substances, for example, wax D from Mycobacterium tuberculosis, substances found in Corynebacterium parvum, or Bordetella pertussis, Freund's adjuvant (DIFCO), alum adjuvant (Alhydrogel), MF-50 (Chiron) Novasomes™, or micelles, among others.

Suitable excipients for liquid formulation include water or saline, suspending agents such as sodium carboxymethylcellulose, methylcellulose, hydroxypropylmethyl-cellulose, sodium alginate, polyvinylpyrrolidone, gum tragacanth, and gum acacia; dispersing or wetting agents such as lecithin, condensation products of an alkylene oxide with fatty acids (e.g., polyoxyethylene stearate), condensation products of ethylene oxide with long chain aliphatic alcohols (e.g., heptadecethyleneoxy-cetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyoxyethylene sorbitan monooleate).

Suitable excipients for solid formulations include calcium carbonate, sodium carbonate, lactose, calcium phosphate, or sodium phosphate; granulating and disintegrating agents such as maize starch, or alginic acid; binding agents such as starch, gelatin, or acacia; and lubricating agents such as magnesium stearate, stearic acids, or talc, and inert solid diluents such as calcium carbonate, calcium phosphate, or kaolin.

Other suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

Certain derivatives of the compound of propofol or the psychotropic drug of the invention which may have little or no pharmacological activity themselves can, when administered into or onto the body, be converted into compounds having the desired activity, for example, by hydrolytic cleavage. Such derivatives are referred to as “prodrugs.” Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association). Other prodrugs in accordance with the invention can, for example, be produced by replacing appropriate functionalities present in the compound drug with certain moieties known to those skilled in the art as ‘pro-moieties’ as described, for example, in “Design of Prodrugs” by H Bundgaard (Elsevier, 1985).

Also included within the scope of the invention are metabolites of propofol or the psychotropic drug that are compounds formed in vivo upon administration of these drugs. Some examples of metabolites in accordance with the invention include: (i) where the compound contains a methyl group, an hydroxymethyl derivative thereof (—CH₃→CH₂OH); (ii) where the compound contains a tertiary amino group, a secondary amino derivative thereof (—NR¹R²→—NHR¹ or →—NHR²); (iii) where the compound of formula (I) contains a secondary amino group, a primary derivative thereof (—NHR¹→—NH₂); (iv) where the compounds contains a phenyl moiety, a phenol derivative thereof (—Ph→—PhOH); and (v) where the compounds contains an amide group, a carboxylic acid derivative thereof.

The invention also provides a combination therapy pack or kit comprising one or more containers filled with one or more compositions comprising a propofol composition and one or more psychotropic drug. The kits are provided for the treatment or amelioration of symptoms of disease and disorders related to a neuropsychiatric disease. The kit comprises instructions for treating the neuropsychiatric disease or disorder as described supra in a subject and one or more of the following components: 1) a first composition comprising a propofol composition; 2) a second composition comprising one or more psychotropics; 3) instruction to use the kit, and 4) optionally associated with such container(s) can be a notice in the form prescribed by a governmental agency regulating the manufacture, use or sale of pharmaceuticals or biological products, which notice reflects approval by the agency of manufacture, use or sale for human administration.

If a particular component is not included in the kit, the kit can optionally comprise information on where to obtain the missing component, for example an order form or uniform resource locator for the internet specifying a website where the component can be obtained.

The instructions provided with the kit describe the practice of the methods of the invention as described above, and the route of administration and effective concentration and the dosing regimen for each of the compositions provided therein.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. The contents of all references, patents and published patent applications cited throughout this application are expressly incorporated herein by reference.

### EXAMPLE 1

### Effective Concentration of Propofol and Mode of Administration

According to the present invention, a method is provided for rapid or instantaneous treatment of depression, suicidality, Post-Traumatic Stress Disorder (PTSD), Asperger's syndrome, Pervasive Developmental Disorder, Social Phobia, anhedonia and Negative Symptoms of Schizophrenia, and anorexia, by administering an effective subanesthetic or anesthetic dose of propofol or fospropofol disodium.

Fospropofol disodium (C-13, H-19, Na-2, 0-2, P, MW=332.240261) is a water-soluble prodrug of the intravenous anesthetic propofol (2,6 diisopropylphenol). It is converted to propofol in the liver and is a short acting hypnotic, sedative, and anesthetic agent. It can be administered in an aqueous solution and may be preferred to propofol as it does not cause injection-site pain.

Fospropofol was administered by several routs including intravenously, or sublingually, buccally, intra-nasally or by rectal suppository or rectal gel; or orally by tablet, capsule, rapid disintegrating formulation, powder, chewable, suspension, liquid or effervescent granule;

Examples of appropriate dosages of propofol are:
1. 0.6-5.0 mg/kg of propofol by buccal administration (bioavailability estimates of ˜50%)
2. 0.3-2.5 mg/kg of propofol by intravenous administration (bioavailability estimates of ˜100%)
3. 0.32-2.7 mg/kg of propofol by intramuscular administration (bioavailability estimates of ˜93%)
4. 0.6-10 mg/kg of propofol by intranasal administration (bioavailability estimates of ˜25-50%)
5. 1.1-19 mg/kg of propofol by oral administration (bioavailability estimates of ˜13-27%)
6. 1.2-10 mg/kg of propofol by rectal administration (bioavailability estimates of ˜25%)
7. 3-25 mg/kg of propofol by transdermal administration (bioavailability estimates of ˜10%)

### EXAMPLE 2

### Combination Therapy

Propofol or fospropofol disodium administered on its own or in combination with other psychotropic medications (e.g., antidepressant medication, antipsychotic medication, mood stabilizing medication, stimulants, hypnotic agent, benzodiazepines, ketamine and other psychotropics. The combination therapy is tailored to specific patient's needs and their specific underlying neuropsychiatric disease or disorder. The specific dosage and mode of administration is designed based on the clinical and physiological characteristics of each specific patient. For example, the mode of administration can be sublingually, buccally, intra-nasally or by rectal suppository or rectal gel. The inventive combination therapy is useful in the treatment of treatment resistant depression, the rapid treatment of depression, suicidality, Posttraumatic Stress Disorder (PTSD), and disorders characterized by social discomfort and lack of initiative in social interaction such as Asperger's syndrome. An example of psychotropic drug candidates for combination with propofol or fospropofol are the following: Doses are examples and not intended to exclude other dosages required in treatment of the aforementioned diseases and are provided herein by way of example and not limitation.

### SSRIs

Selective serotonin reuptake inhibitors (SSRIs) are common medications. They fight depression symptoms by decreasing serotonin blockers in the brain. They're the most commonly prescribed class of antidepressants. They come in the form of:
1. sertraline (Zoloft)—25-200 mg/day Oral
2. fluoxetine (Prozac)—10 or 20 mg/day Oral
3. citalopram (Celexa)—20-40 mg/day Oral
4. escitalopram (Lexapro)—10-20 mg/day Oral
5. paroxetine (Paxil, Pexeva)—20-50 mg/day (25-62.5 mg/day for extended release tablet)
6. fluvoxamine (Luvox) 100-300 mg/day for OCD Oral (not approved for pediatric treatment of depression yet)
7. trazodone (Oleptro)—150-375 mg/day extended release, 150-400 mg/day immediate release (the maximum is 600 mg/day for outpatients though.)

### SNRIs and Related Compounds

Serotonin and norepinephrine reuptake inhibitors (SNRIs) help improve serotonin and norepinephrine levels in the brain. Options include:
1. Desvenlafaxine (Pristiq)—50-400 mg/day oral
2. Duloxetine (Cymbalta)—20-120 mg/day oral
3. Venlafaxine (Effexor XR)—75/375 mg/day for extended and immediate release tablets
4. Atomoxetine—40 mg to 100 mg once a day or in divided doses, oral
5. Mirtazapine 15-45 mg orally once a day

### TCAs

Tricyclic antidepressants (TCAs) are often prescribed when SSRIs or other antidepressants don't work. TCAs can cause constipation, dry mouth, and fatigue. More serious side effects include low blood pressure, irregular heart rate, and seizures. TCAs are available as:
1. Amitriptyline
   a. Recommended oral geriatric dose
      i. 10 mg orally 3 times a day with 20 mg given at bedtime
   b. Oral dose adults
      i. 75 mg to 300 mg per day in divided doses, or 40 to 100 mg once orally at bedtime
2. Amoxapine
   a. Oral dose adults
      i. 100 mg divided into two does per day to 600 mg per day
3. Clomipramine (Anafranil)
   a. Oral drug dose adults
      i. An initial dose of 25 mg once per day before bedtime, with maintenance doses ranging from 100 mg to 250 mg once per day
4. Desipramine (Norpramin)
   a. Oral drug dose adults
      i. 100 mg to 300 mg per day, may be lowered when combined with other drugs that inhibit CYP450 2D6
5. Doxepin
   a. Oral drug dose adults
      i. Initial drug dose ranges from 25 mg to 150 mg per day divided into one to three doses, depending on the severity of the depression. The maintenance dose ranges from 25 mg to 300 mg per day divided into one to three doses, depending on the severity of the depression.
6. Imipramine (Tofranil)
   a. Oral tablet dose adults
      i. For hospital patients, the initial dose is usually 100 mg per day while the maintenance dose ranges from 100 mg per day to 300 mg per day. For outpatients, the initial dose is usually 75 mg per day and the maintenance dose ranges from 50 mg per day to 200 mg per day.
   b. Oral capsule dose adults
      i. For hospital patients, the initial dose is usually 100 mg to 150 per day while the maintenance dose ranges from 75 mg per day to 300 mg per day. For outpatients, the initial dose is usually 75 mg per day and the maintenance dose ranges from 75 mg per day to 200 mg per day.
   c. Oral geriatric dose tablets
      i. The initial dose ranges from 30 mg per day to 40 mg per day. The maximum dose is 100 mg per day.
   d. Oral geriatric dose capsules
      i. The initial dose ranges from 25 mg per day to 50 mg per day. The maximum dose is 100 mg per day.
7. Nortriptyline (Pamelor)
   a. Oral drug dose adults
      i. The dose ranges from 25 mg three- or four-times day to 150 mg per day.
   b. Oral geriatric drug dose
      i. The dose ranges from 30 mg to 50 mg per day in divided doses.
8. Protriptyline (Vivactil)
   a. Oral drug dose adults
      i. The dose ranges from 15 mg three- or four-times day to 40 mg three or four times per day. The maximum dose is 60 mg per day.
   b. Oral geriatric drug dose
      i. The usual dose is 5 mg three times per day, though this may be exceeded
9. Trimipramine (Surmontil)
   a. Oral drug dose adults
      i. For outpatients, the initial dose is usually 75 mg per day in divided dose while the maintenance dose ranges from 50 mg per day to 200 mg per day. For hospitalized patients, the initial dose is usually 100 mg per day in divided dose and the maintenance dose ranges from 200 mg per day to 300 mg per day.
   b. Oral geriatric drug dose
      i. The initial dose is usually 50 mg per day administered once at bedtime. The maintenance dose is usually 100 mg per day administered once at bedtime.

### MAOIs

Monoamine oxidase inhibitors (MAOIs) are older drugs that treat depression by preventing the breakdown of norepinephrine, dopamine, and serotonin. They're more difficult for patients to take than most other antidepressants because they interact with many prescription drugs, nonprescription drugs, and foods. They have many adverse effects and can't be combined with other antidepressants or stimulants. MAOIs are rarely a doctor's first choice of drug to prescribe. MAOIs include:
1. Isocarboxazid (Marplan) 20-60 mg/day oral
2. Phenelzine (Nardil)—60-90 mg/day oral
3. Selegiline (Emsam), a transdermal patch 6-12 mg/24 hours
4. Tranylcypromine (Parnate)—20-60 mg/day oral

### Antipsychotic Medication

1. Lurasidone—20 mg to 160 mg per day, oral
2. Quetiapine—150 mg to 300 mg per day, extended release tablets administered orally
3. Brexpiprazole—0.5-4 mg/day

### Anti-Convulsants

1. Lamotrigine 25 mg-200 mg daily
   Drugs acting on glutaminergic receptors and transporters including NMDA, ionotropic receptors, AMPA, kainate metabotropic receptor and GluN2C glycine binding site Agonists (Partial and full), Co-agonists, Antagonists (Partial and full), Allosteric and other Modulators; and glutamate transporters, including but not limited to, Ketamine administered intravenously, subcutaneously, intramuscularly, intranasally by spray, powder or other means, intra-pulmonary, intra-rectal or by any route
1. GLYX13 Rapastinel
2. NRX 1074
3. Memantine
4. CERC 301
5. Traxoprodil
6. D Cycloserine

All references, including publications, patent applications, and patents, cited herein are hereby incorporated by reference to the same extent as if each reference were individually and specifically indicated to be incorporated by reference and were set forth in its entirety herein.

The use of the terms “a,” “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language provided herein, is intended merely to further illustrate the content of the disclosure and does not pose a limitation on the scope of the claims. No language in the specification should be construed as indicating any non-claimed element as essential to the practice of the present disclosure.

The individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

What is claimed is:

1. A composition for treating or ameliorating symptoms of a neuropsychiatric disease in a subject in need thereof comprising: (a) a therapeutically effective amount of propofol; and (b) a therapeutically effective amount of one or more psychotropic drug and a pharmaceutically acceptable carrier or diluent.

2. The composition according to claim 1, wherein said neuropsychiatric disease comprising depression, treatment resistant depression, suicidality, Posttraumatic Stress Disorder, Asperger's Syndrome, pervasive developmental disorder, social phobia, anxiety, anorexia, anhedonia and schizophrenia.

3. The composition according to claim 1, wherein the one or more psychotropic drug comprises Selective Serotonin Reuptake Inhibitors (SSRIs), Serotonin and Norepinephrine Reuptake Inhibitors (SNRIs), Tricyclic Antidepressants, Monoamine Oxidase Inhibitors, antipsychotic medication, anticonvulsants, mood stabilizing medication, stimulants, hypnotic agent, benzodiazepines, and ketamine.

4. The composition according to claim 2, wherein said depression is treated instantaneously and rapidly.

5. The composition of claim 1 where said propofol comprises propofol analogues, derivatives, salts and racemic mixtures, prodrugs and/or metabolites of propofol or fospropofol disodium. or any combination thereof.

7. The composition according to claim 1, wherein said propofol and said one or more psychotropic drugs are formulated in one or different solutions.

8. The composition according to claim 1, wherein said propofol and said one or more psychotropic drug are in a dry or a liquid formulation.

9. The composition according to claim 1, wherein said propofol and said one or more psychotropic drug are administered contemporaneously or at different time intervals.

10. A method for treating or ameliorating symptoms of a neuropsychiatric disease comprising administrating to a subject in need thereof: (a) a therapeutically effective amount of propofol; and (b) a therapeutically effective amount of one or more psychotropic drug and a pharmaceutically acceptable carrier or diluent.

11. The method according to claim 10, wherein said neuropsychiatric disease comprising severe anxiety, tremors, depression, dysthymia, mania, hypomania, panic disorder, phobias, suicidality, posttraumatic stress disorder, Asperger's Syndrome, pervasive developmental disorder, Social Phobia, anorexia, anhedonia and schizophrenia.

12. The method according to claim 10, wherein the one or more psychotropic drug comprises Selective Serotonin Reuptake Inhibitors (SSRIs), Serotonin and Norepinephrine Reuptake Inhibitors (SNRIs), Tricyclic Antidepressants, Monoamine Oxidase Inhibitors, antipsychotic medication, anticonvulsants, mood stabilizing medication, stimulants, hypnotic agent, benzodiazepines, and ketamine.

13. The method according to claim 11, wherein said depression is treated instantaneously and rapidly.

14. The method according to claim 10, wherein said propofol comprises propofol analogues, derivatives, salts and racemic mixtures, prodrugs and/or metabolites of propofol or fospropofol disodium. or any combination thereof.

15. The method according to claim 10, wherein said propofol and said one or more psychotropic drugs are formulated in one or different solutions.

16. The method according to claim 10, wherein said propofol and said one or more psychotropic drug are in a dry or a liquid formulation.

17. The method according to claim 10, wherein said propofol and said one or more psychotropic drug are administered successively at different time intervals or contemporaneously.

18. The method according to claim 10, wherein said propofol, and said one or more psychotropic drug, or both are administered orally, parenterally, transdermally, intranasally, or intravenously.

19. The method of claim 10, wherein said propofol and said one or more psychotropic drug are administered in a time-released manner.

20. A method for treating or ameliorating symptoms of a neuropsychiatric disease in a subject comprising administering to a subject in need thereof a therapeutically effective amount of propofol, or analogues, derivatives, salts and racemic mixtures of propofol or fospropofol.

21. A kit for treating or ameliorating one or more symptoms of a neuropsychiatric disease comprising:
a) a first composition comprising a propofol composition;
b) a second composition comprising one or more psychotropic drugs; and
c) instructions for the use of the first and second compositions.

22. A method of achieving an enhanced therapeutic effectiveness of a psychotropic drug in a subject in need thereof comprising administrating one or more psychotropic drug in combination with a propofol composition, wherein the effective therapeutic concentration of said one or more psychotropic drug is reduced by about 10-100 fold when administered with said propofol composition as compared to a control group that did not receive said propofol composition.

23. The method of claim 22, wherein reduced dosing of psychotropic drug results in prevention or amelioration of one or more side effects resulted from the prolonged use of said one or more psychotropic drug, wherein said side effects comprise addiction, dysphoria, anxiety, panic, impairment of memory, reductions in psychomotor and cognitive performance, disordered perception of the passage of time, euphoria, schizophrenic psychosis, tiredness, dizziness, tachycardia, orthostatic hypotension, dry mouth, reduced lacrimation, muscle relaxation, increased appetite, potential irreversible cognitive impairments, or any combination thereof.
